(19) United States
(12) Patent Application Publication
(10) Pub. No.: US 2018/0002295 A1
(43) Pub. Date: Jan. 4, 2018

(54) Compositions and Methods for Treating Anxiety and Compulsive Behavior

(71) Applicant: University of Iowa Research Foundation, Iowa City, IA (US)

(72) Inventors: Andrew A. PIEPER, Iowa City, IA (US); Gregory FRIESTAD, Iowa City, IA (US)

(21) Appl. No.: 15/545,283

(22) PCT Filed: Jan. 25, 2016
(86) PCT No.: PCT/US2016/014732

§ 371 Date: Jul. 20, 2017

Related U.S. Application Data
(60) Provisional application No. 62/107,473, filed on Jan. 25, 2015.

(51) Int. Cl. C07D 239/84

(52) U.S. Cl. CPC C07D 239/84

(57) ABSTRACT

Provided herein is compositions and methods for treating anxiety, obsession and/or pathologically compulsive behavior associated with various neuropsychiatric diseases.

### CROSS REFERENCE TO RELATED APPLICATIONS

This application claims priority to and the benefit of U.S. Provisional Application No. 62/107,473, filed Jan. 25, 2015, the entire disclosure of which is incorporated herein by reference in its entirety.

### FIELD

This disclosure relates to compounds and their use in treating anxiety and pathologically compulsive behavior associated with various neuropsychiatric diseases.

### BACKGROUND

Anxiety Disorders affect about 40 million American adults age 18 years and older (about 18%) in a given year, causing them to be filled with fearfulness and uncertainty. Unlike the relatively mild, brief anxiety caused by a stressful event (such as speaking in public or a first date), anxiety disorders last at least 6 months and can get worse if they are not treated. Common types of anxiety disorders include panic disorder, obsessive-compulsive disorder (OCD), post-traumatic stress disorder (PTSD), social phobia (or social anxiety disorder), specific phobias, and generalized anxiety disorder. Anxiety disorders account for approximately one-third of US mental health expenditures, with an annual estimated cost of $42.3 billion in 1990; for the same year OCD costs were estimated at $2.1 billion annually.

OCD is a common and debilitating psychiatric illness that affects 2-3% of the U.S. population and is the fourth psychiatric disorder in terms of incidence. OCD is characterized by a combination of persistently intrusive thoughts, repetitive actions and excessive anxiety, which together impair functioning. Intrusive thoughts constitute obsessions commonly involving preoccupation with contamination, doubting, symmetry, religious or sexual themes, or a premonition that a bad outcome will result if a specific ritual is not executed. Repetitive actions are compulsions usually linked to these thoughts, which typically comprise ritualistic physical behaviors, such as washing, cleaning, checking, repeating, counting, arranging and hoarding. Tragically, there is a void of specific and effective treatments for patients with pathologically compulsive behavior, as its neurobiological basis is poorly understood. Current first-line therapy includes selective serotonin reuptake inhibitors, such as fluoxetine, which are marginally effective and may be associated with undesirable side effects, with a rate of treatment resistance of 40%. Patients are also often afflicted with equivalently poorly understood and difficult to treat incapacitating obsessive-compulsive (OC)-spectrum disorders that share clinical features with OCD, including tics, Tourette's syndrome, trichotillomania, body dysmorphic disorder and hypochondriasis. Data from the National Comorbidity Survey Replication (NCS-R) show that 90% of the patients with a lifetime diagnosis of OCD meet criteria for another DSM-IV disorder, most frequently other anxiety disorders or mood disorders.

Pathologically compulsive behavior is a highly prevalent and difficult to treat component of many forms of neuropsychiatric disease, including OCD and OC-spectrum conditions. Current treatments for compulsive behavior or anxiety are confined to non-pharmacologic behavioral therapy, or two pharmacologic approaches (benzodiazepines or selective serotonin reuptake inhibitors). All modes of treatment currently available are insufficient to treat the large number of afflicted patients, and existing pharmacologic approaches are also complicated by side effects that often prohibit treatment. Furthermore, little is known of the underlying basic science mechanisms of pathologically compulsive behavior.

Thus, a need exists for new pharmacologic treatments for pathologically compulsive behavior and/or anxiety, as well as molecular tools to investigate the underlying pathophysiology.

### SUMMARY

This disclosure relates to, in one aspect, compounds of formula (I) or (II) (also referred to as “P5C6 class” of compounds), or a pharmaceutically acceptable salt or prodrug thereof:

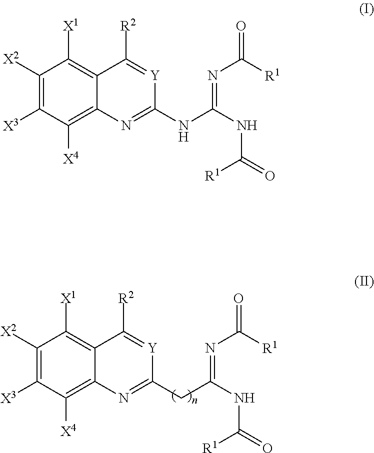

wherein:
X¹, X², X³, and X⁴ are each independently selected from: hydrogen; halo; hydroxyl; C₁₋₆ (e.g., C₁₋₃) alkoxyl optionally substituted with 1 or more hydroxyl, cyano and/or halo; C₁₋₆ (e.g., C₁₋₃) alkyl carbonyl; C₁₋₆ (e.g., C₁₋₃) alkoxycarbonyl; cyano; nitro; amine; R^{A} and R^{B};
   wherein R^{A} at each occurrence is independently selected from C₁₋₆ (e.g., C₁₋₃) alkyl, C₂₋₆ (e.g., C₂₋₃) alkenyl and C₂₋₆ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) thioalkoxyl, C₁₋₆ (e.g., C₁₋₃) alkyl carbonyl, C₁₋₆ (e.g., C₁₋₃) alkoxycarbonyl, cyano, nitro, and/or amine;
   wherein R^{B} at each occurrence is independently selected from C₃₋₁₂ cycloalkyl, C₂₋₆ heterocyclyl, C₆₋₁₂ aryl and C₄₋₁₂ heteroaryl, each optionally substituted with 1 or more halo; hydroxyl; C₁₋₆ alkyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₄₋₁₂ heteroaryl, and/or amine; C₂₋₆ (e.g., C₂₋₃) alkoxycarbonyl; cyano; nitro; azide; amine; C₃₋₁₂ cycloalkyl; C₂₋₆ heterocyclyl; C₆₋₁₂ aryl; and/or C₄₋₁₂ heteroaryl; wherein each of the substituents C₃₋₁₂ cycloalkyl, C₂₋₆ heterocyclyl. C₆₋₁₂ aryl and C₄₋₁₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) thioalkoxyl, C₁₋₆ (e.g., C₁₋₃) alkoxycarbonyl, cyano, nitro and/or amine;
R¹ and R² at each occurrence, are each independently selected from C₁₋₁₂ alkyl, C₂₋₁₂ alkenyl or C₂₋₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆ alkoxycarbonyl, cyano, nitro and/or amine; wherein the C₁₋₆ alkoxyl, C₁₋₆ thioalkoxyl, C₁₋₆ alkyl carbonyl, and C₁₋₆ alkoxycarbonyl groups each are optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆ alkoxycarbonyl, cyano, nitro and/or amine;
Y is CH or N; and
n is an integral selected from 1-12.

In some embodiments, at least one of X¹, X², X³, and X⁴ is not hydrogen. For example, X² is not hydrogen. In certain embodiments, X² is ethoxyl. In certain embodiments, X² is not ethoxyl. The two R¹ groups in (I) or (II) can be the same or different. In some embodiments, one or both of R¹ is not ethyl. In some embodiments, one or both of R¹ is ethyl.

The compound, in some embodiments, can be (+) or (−) (dextrorotatory) when in the presence of plane polarized light. In some embodiments, the (+) (dextrorotatory) compound can be substantially free of (e.g., containing less than about 5% of, less than about 2% of, less than about 1%, less than about 0.5%) a compound that is (levororotatory). In some embodiments, the (−) (levororotatory) compound can be substantially free of (e.g., containing less than about 5% of, less than about 2% of, less than about 1%, less than about 0.5%) a compound that is (+) (dextrorotatory).

The compound of the present disclosure can include any one or more compounds selected from:

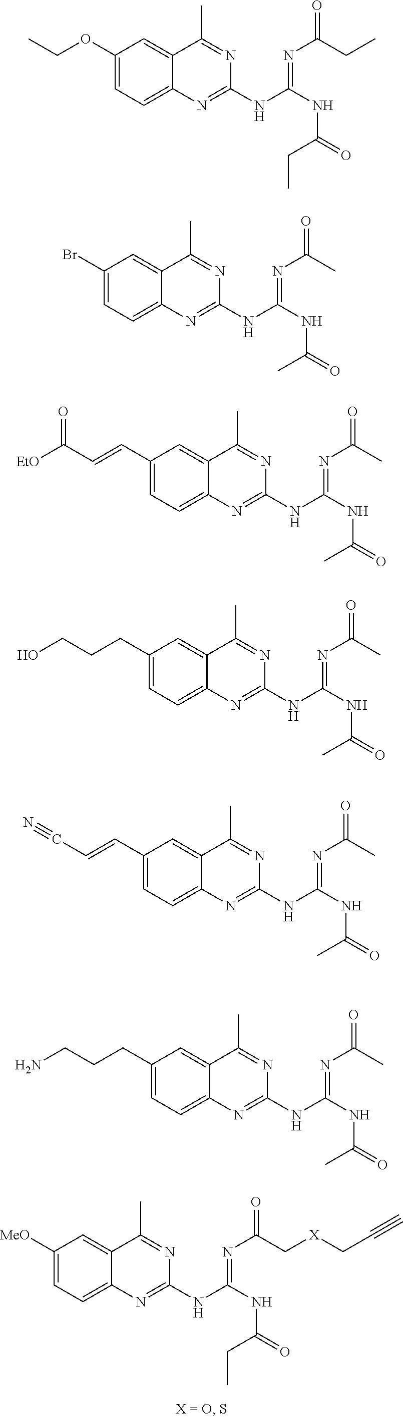

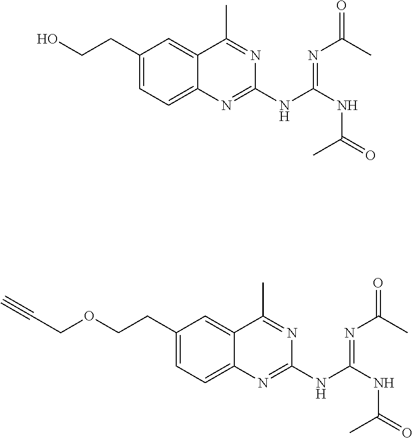 [SMILES: CC(=O)/N=C(\NC(C)=O)Nc1nc(C)c2cc(CCO)ccc2n1 ; C#CCOCCc1ccc2nc(N/C(=N/C(C)=O)NC(C)=O)nc(C)c2c1]

or a salt (e.g., a pharmaceutically acceptable salt) or a prodrug thereof.

In certain embodiments, the compound or salt is P5C6 having the following structure:

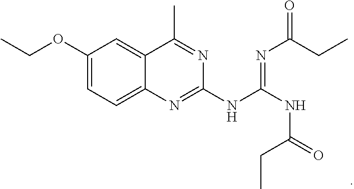 [SMILES: CCOc1ccc2nc(N/C(=N/C(=O)CC)NC(=O)CC)nc(C)c2c1]

In some embodiments, the above compounds (such as a formula (I) or (II) compound), or a pharmaceutically acceptable salt or prodrug thereof, can be used (e.g., in the manufacture of a medicament) to treat anxiety, obsession, compulsive behavior and/or a disease such as anxiety disorder or a neuropsychiatric disease, including but not limited to major depression, schizophrenia, autism, autism spectrum disorder, obsessive compulsive personality disorder, bipolar disorder, generalized anxiety disorder, social anxiety disorder, pediatric autoimmune neuropsychiatric disorders associated with streptococcal infections (PANDAS), pediatric acute-onset neuropsychiatric syndrome (PANS), anorexia nervosa, bulimia nervosa, Tourette syndrome, Asperger syndrome, body dysmorphic disorder, eating disorders, panic disorder, social phobia, Sydenham's chorea, Parkinson's disease, Huntington's disease, hoarding disorder, tic disorder, trichotillomania, dementia, Alzheimer's disease, attention deficit hyperactivity disorder, dermatillomania, onychophagia, and drug addiction.

Also disclosed herein is a compound of formula (I) or (II), or a pharmaceutically acceptable salt or prodrug thereof, for use in the treatment of one or more of the above diseases.

A further aspect relates to use of the compound or salt or prodrug disclosed herein for the manufacture of a medicament for the treatment of one or more of the above diseases.

Also provided herein is a pharmaceutical composition comprising a compound of formula (I) or (II), or a pharmaceutically acceptable salt or prodrug thereof, and a pharmaceutically acceptable carrier.

Provided herein, in another aspect, is a method for treating one or more of the above diseases or conditions. The method includes administering an effective amount of a compound of formula (I) or (II) or a pharmaceutically acceptable salt or prodrug thereof, to a patient in need thereof.

The present disclosure features compositions (e.g., pharmaceutical compositions) that include a compound of formula (I) or (II), as well as methods of making, identifying, and using such compounds. Other features and advantages are described in, or will be apparent from, the present specification and accompanying drawings.

### BRIEF DESCRIPTION OF THE DRAWINGS

FIG. 1 shows SAPAP3-deficient (sapap3−/−) mice, a rodent model of pathologically compulsive behavior, exhibit significantly increased basal and spray-induced grooming relative to wild type littermates. Intraperitoneal (i.p.) treatment of sapap3−/− mice with fluoxetine, or intracerebroventricular (i.c.v.) administration of P5C6, however, normalized grooming. Statistics were conducted with Student's t test.

FIG. 2A shows a general synthetic route to N,N-diacylguanidinoquinazolines (Webb, 2003).

FIG. 2B shows readily available substituted anilines as alternative precursors.

FIG. 3 shows a general synthetic route.

FIG. 4 shows exemplary modifications for bioconjugate synthesis and a representative P5C6 biotin conjugate.

FIG. 5 shows examples of P5C6 analog synthesis. Yields are unoptimized.

FIGS. 6A-6D show exemplary P5C6 analogs and representative synthetic routes.

### DETAILED DESCRIPTION

### Definitions

Unless defined otherwise, all technical and scientific terms used herein have the meaning commonly understood by one of ordinary skill in the art to which the disclosure pertains. Specific terminology is defined below.

As used in the specification and the appended claims, the singular forms “a,” “an,” and “the” include plural referents unless the context clearly dictates otherwise.

As used herein, the term “about” means within 20%, more preferably within 10% and most preferably within 5%.

“An effective amount” refers to an amount of a compound that confers a therapeutic effect (e.g., treats, e.g., controls, relieves, ameliorates, alleviates, or slows the progression of; or prevents, e.g., delays the onset of or reduces the risk of developing, a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/kg to about 1000 mg/kg, (e.g., from about 0.1 mg/kg to about 100 mg/kg, from about 1 mg/kg to about 100 mg/kg). Effective doses will also vary depending on route of administration, as well as the possibility of co-usage with other agents.

The following definitions of various groups or substituents are used, unless otherwise described. Specific and general values listed below for radicals, substituents, and ranges, are for illustration only; they do not exclude other defined values or other values within defined ranges for the radicals and substituents. Unless otherwise indicated, alkyl, alkoxy, alkenyl, and the like denote both straight and branched groups.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substituents. Examples of alkyl groups include without limitation methyl, ethyl, n-propyl, isopropyl, and tert-butyl.

As used herein, the term “straight chain Cₙ₋ₘ alkylene,” employed alone or in combination with other terms, refers to a non-branched divalent alkyl linking group having n to m carbon atoms. Any atom can be optionally substituted, e.g., by one or more substituents. Examples include methylene (i.e., —CH₂—).

The term “haloalkyl” refers to an alkyl group, in which at least one hydrogen atom is replaced by halo. In some embodiments, more than one hydrogen atom (e.g., 2, 3, 4, 5, 6, 7, 8, 9, 10, 11, 12, 13, or 14) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

As referred to herein, the term “alkoxy” refers to a group of formula —O(alkyl). Alkoxy can be, for example, methoxy (—OCH₃), ethoxy, propoxy, isopropoxy, butoxy, iso-butoxy, sec-butoxy, pentoxy, 2-pentoxy, 3-pentoxy, or hexyloxy. Likewise, the term “thioalkoxy” refers to a group of formula —S(alkyl). Finally, the terms “haloalkoxy” and “halothioalkoxy” refer to —O(haloalkyl) and —S(haloalkyl), respectively. The term “sulfhydryl” refers to —SH. As used herein, the term “hydroxyl,” employed alone or in combination with other terms, refers to a group of formula —OH.

The term “aralkyl” refers to an alkyl moiety in which an alkyl hydrogen atom is replaced by an aryl group. One of the carbons of the alkyl moiety serves as the point of attachment of the aralkyl group to another moiety. Any ring or chain atom can be optionally substituted e.g., by one or more substituents. Non-limiting examples of “aralkyl” include benzyl, 2-phenylethyl, and 3-phenylpropyl groups.

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent.

The term “alkynyl” refers to a straight or branched hydrocarbon chain containing the indicated number of carbon atoms and having one or more carbon-carbon triple bonds. Alkynyl groups can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “heterocyclyl” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. The heteroatom or ring carbon can be the point of attachment of the heterocyclyl substituent to another moiety. Any atom can be optionally substituted, e.g., by one or more substituents. Heterocyclyl groups can include, e.g., tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected R^{a}” would include (but not be limited to) tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include, e.g., dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. Cycloalkenyl moieties can include, e.g., cyclohexenyl, cyclohexadienyl, or norbornenyl.

As used herein, the term “cycloalkylene” refers to a divalent monocyclic cycloalkyl group having the indicated number of ring atoms.

As used herein, the term “heterocycloalkylene” refers to a divalent monocyclic heterocyclyl group having the indicated number of ring atoms.

The term “aryl” refers to an aromatic monocyclic, bicyclic (2 fused rings), or tricyclic (3 fused rings), or polycyclic (>3 fused rings) hydrocarbon ring system. One or more ring atoms can be optionally substituted, e.g., by one or more substituents. Aryl moieties include, e.g., phenyl and naphthyl.

The term “heteroaryl” refers to an aromatic monocyclic, bicyclic (2 fused rings), tricyclic (3 fused rings), or polycyclic (>3 fused rings) hydrocarbon groups having one or more heteroatom ring atoms independently selected from O, N (it is understood that one or two additional groups may be present to complete the nitrogen valence and/or form a salt), or S. One or more ring atoms can be optionally substituted, e.g., by one or more substituents. Examples of heteroaryl groups include, but are not limited to, 2H-pyrrolyl, 3H-indolyl, 4H-quinolizinyl, acridinyl, benzo[b]thienyl, benzothiazolyl, O-carbolinyl, carbazolyl, coumarinyl, chromenyl, cinnolinyl, dibenzo[b,d]furanyl, furazanyl, furyl, imidazolyl, imidizolyl, indazolyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, and xanthenyl.

The terms “arylcycloalkyl” and “arylheterocyclyl” refer to bicyclic, tricyclic, or other polycyclic ring systems that include an aryl ring fused to a cycloalkyl and heterocyclyl, respectively. Similarly, the terms “heteroarylheterocyclyl,” and “heteroarylcycloalkyl” refer to bicyclic, tricyclic, or other polycyclic ring systems that include a heteroaryl ring fused to a heterocyclyl and cycloalkyl, respectively. Any atom can be substituted, e.g., by one or more substituents. For example, arylcycloalkyl can include indanyl; arylheterocyclyl can include 2,3-dihydrobenzofuryl, 1,2,3,4-tetrahydroisoquinolyl, and 2,2-dimethylchromanyl.

The descriptors “C═O” or “C(O)” or “carbonyl” refers to a carbon atom that is doubly bonded to an oxygen atom. “Alkyl carbonyl” has a common formula of R—C(O)— wherein R may be C₁₋₁₂ alkyl, C₂₋₁₂ alkenyl, C₂₋₁₂ alkynyl, C₃₋₁₂ cycloalkyl, C₆₋₁₂ aryl, C₄₋₁₂ heteroaryl, or C₃₋₁₂ heterocyclyl.

The term “oxo” refers to double bonded oxygen which can be a substituent on carbon or other atoms. When oxo is a substituent on nitrogen or sulfur, it is understood that the resultant groups has the structures N→O⁻ and S(O) and SO₂, respectively.

As used herein, the term “cyano,” employed alone or in combination with other terms, refers to a group of formula —CN, wherein the carbon and nitrogen atoms are bound together by a triple bond. The term “azide” refers to a group of formula —N₃. The term “nitro” refers to a group of formula —NO₂. The term “amine” includes primary (—NH₂), secondary (—NHR), tertiary (—NRR′), and quaternary (—N⁺RR′R″) amine having one, two or three independently selected substituents such as straight chain or branched chain alkyl, alkenyl, alkynyl, cycloalkyl, aryl, heteroaryl, heterocycle, and the like.

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

The terms “treating” and “treatment” as used herein refer to reduction in severity and/or frequency of symptoms, elimination of symptoms and/or underlying cause, and improvement or remediation of damage.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug administration.

As used herein, the term “patient” or “individual” or “subject” refers to any person or mammalian subject for whom or which therapy is desired, and generally refers to the recipient of the therapy to be practiced according to the disclosure. The term “mammal” includes organisms, which include mice, rats, cows, sheep, pigs, rabbits, goats, horses, monkeys, dogs, cats, and humans.

The term “substituent” refers to a group “substituted” on, e.g., an alkyl, haloalkyl, cycloalkyl, heterocyclyl, heterocycloalkenyl, cycloalkenyl, aryl, or heteroaryl group at any atom of that group, replacing one or more hydrogen atom therein. In one aspect, the substituent(s) on a group are independently any one single, or any combination of two or more of the permissible atoms or groups of atoms delineated for that substituent. In another aspect, a substituent may itself be substituted with any one of the above substituents. Further, as used herein, the phrase “optionally substituted” means unsubstituted (e.g., substituted with an H) or substituted. It is understood that substitution at a given atom is limited by valency. Common substituents include halo, C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₄₋₁₂ heteroaryl, C₃₋₁₂ heterocyclyl, C₁₋₁₂alkylsulfonyl, nitro, cyano, —COOR, —C(O)NRR′, —OR, —SR, —NRR′, and oxo, such as mono- or di- or tri-substitutions with moieties such as trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₄₋₁₂ heteroaryl or C₄₋₂₄ heteroarylalkyl. Unless otherwise noted, all groups described herein optionally contain one or more common substituents, to the extent permitted by valency. Further, as used herein, the phrase “optionally substituted” means unsubstituted (e.g., substituted with an H) or substituted. As used herein, the term “substituted” means that a hydrogen atom is removed and replaced by a substituent (e.g., a common substituent). It is understood by one of ordinary skill in the chemistry art that substitution at a given atom is limited by valency. The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

The details of one or more embodiments are set forth in the description below. Other features and advantages of the present disclosure will be apparent from the description and from the claims.

### P5C6 Compounds

Compounds of the present disclosure include, in some embodiments, those of formula (I) or (II), or pharmaceutically acceptable salts or prodrugs thereof:

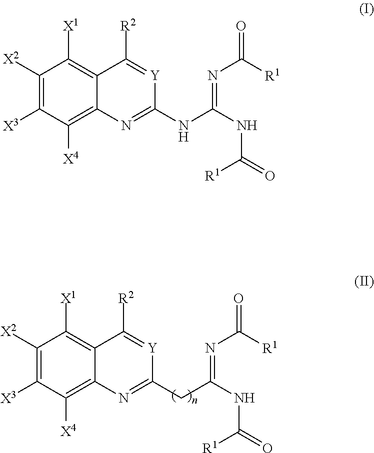

wherein:
X¹, X², X³, and X⁴ are each independently selected from: hydrogen; halo; hydroxyl; C₁₋₆ (e.g., C₁₋₃) alkoxyl optionally substituted with 1 or more hydroxyl, cyano and/or halo; C₁₋₆ (e.g., C₁₋₃) alkyl carbonyl; C₁₋₆ (e.g., C₁₋₃) alkoxycarbonyl; cyano; nitro; amine; R^{A} and R^{B};
   wherein R^{A} is selected from C₁₋₆ (e.g., C₁₋₃) alkyl, C₂₋₆ (e.g., C₂₋₃) alkenyl and C₂₋₆ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) thioalkoxyl, C₁₋₆ (e.g., C₁₋₃) alkyl carbonyl, C₁₋₆ (e.g., C₁₋₃) alkoxycarbonyl, cyano, nitro, and/or amine;
   wherein R^{B} is selected from C₃₋₁₂ cycloalkyl, C₂₋₆ heterocyclyl, C₆₋₁₂ aryl and C₄₋₁₂ heteroaryl, each optionally substituted with 1 or more halo; hydroxyl; C₁₋₆ alkyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₄₋₁₂ heteroaryl, and/or amine; C₂₋₆ (e.g., C₂₋₃) alkoxycarbonyl; cyano; nitro; azide; amine; C₃₋₁₂ cycloalkyl; C₂₋₆ heterocyclyl; C₆₋₁₂ aryl; and/or C₄₋₁₂ heteroaryl; wherein each of the substituents C₃₋₁₂ cycloalkyl, C₂₋₆ heterocyclyl, C₆₋₁₂ aryl and C₄₋₁₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) thioalkoxyl, C₁₋₆ (e.g., C₁₋₃) alkoxycarbonyl, cyano, nitro and/or amine;
R¹ and R² at each occurrence, are each independently selected from C₁₋₁₂ alkyl, C₂₋₁₂ alkenyl or C₂₋₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆ alkoxycarbonyl, cyano, nitro and/or amine; wherein the C₁₋₆ alkoxyl, C₁₋₆ thioalkoxyl, C₁₋₆ alkyl carbonyl, and C₁₋₆ alkoxycarbonyl groups each are optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, alkoxycarbonyl, cyano, nitro and/or amine;
Y is CH or N; and
n is an integral selected from 1-12.

In some embodiments, at least one of X¹, X², X³, and X⁴ is not hydrogen. For example, X² is not hydrogen. In some embodiments, the compound is P5C6. In some embodiments, the compound is not P5C6.

The compound of the present disclosure can include any one or more compounds selected from:

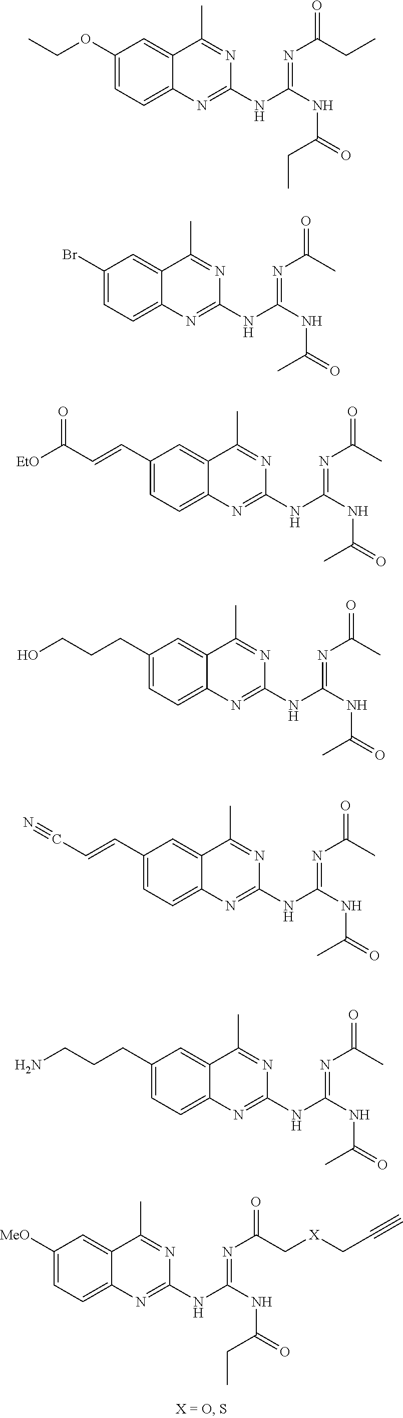

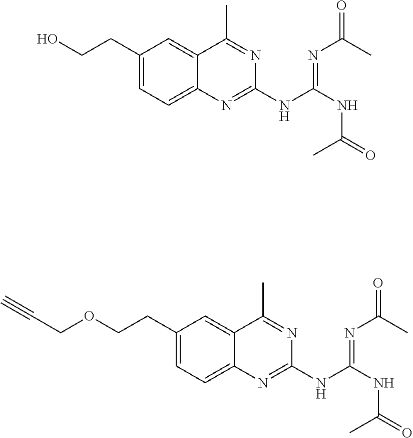 [SMILES: CC(=O)/N=C(\NC(C)=O)Nc1nc(C)c2cc(CCO)ccc2n1 ; C#CCOCCc1ccc2nc(N/C(=N/C(C)=O)NC(C)=O)nc(C)c2c1]

or a salt (e.g., a pharmaceutically acceptable salt) or a prodrug thereof.

In certain embodiments, the compound or salt is P5C6 having the following structure:

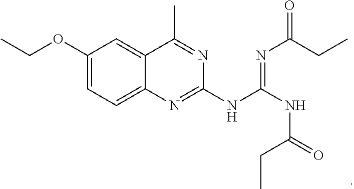 [SMILES: CCOc1ccc2nc(N/C(=N/C(=O)CC)NC(=O)CC)nc(C)c2c1]

### Compound Forms and Salts

The compounds of the present disclosure may contain one or more asymmetric centers and thus occur as racemates and racemic mixtures, enantiomerically enriched mixtures, single enantiomers, individual diastereomers and diastereomeric mixtures. All such isomeric forms of these compounds are expressly included in the present disclosure. The compounds of the present disclosure may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present disclosure. The compounds of the present disclosure may also be represented in multiple tautomeric forms, in such instances, the present disclosure expressly includes all tautomeric forms of the compounds described herein, even though only a single tautomeric form may be represented. All such isomeric forms of such compounds are expressly included in the present disclosure.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that the present disclosure encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The compounds of the present disclosure include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of the present disclosure include those derived from pharmaceutically acceptable inorganic and organic acids and bases. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient.

Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the present disclosure and their pharmaceutically acceptable acid addition salts. Salts derived from appropriate bases include alkali metal (e.g., sodium), alkaline earth metal (e.g., magnesium), ammonium and N-(alkyl)₄⁺ salts. The present disclosure also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxyl groups (e.g. L-arginine, -lysine, -histidine salts).

Lists of suitable salts are found in Remington's Pharmaceutical Sciences, 17th ed., Mack Publishing Company, Easton, Pa., 1985, p. 1418; Journal of Pharmaceutical Science, 66, 2 (1977); and “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8] each of which is incorporated herein by reference in their entireties.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, the active entity. Additional examples include peptidyl derivatives of a compound of the present disclosure.

The present disclosure also includes various hydrate and solvate forms of the compounds.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure.

### Synthesis

The compounds of the present disclosure can be conveniently prepared in accordance with the procedures outlined in the Examples section below. The compounds can also be prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d. ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high performance liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes preparation of the Mosher's ester or amide derivative of the corresponding alcohol or amine, respectively. The absolute configuration of the ester or amide is then determined by proton and/or ¹⁹F NMR spectroscopy. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent compositions can be determined by one skilled in the art.

### Pharmaceutical Compositions

The term “pharmaceutically acceptable carrier” refers to a carrier or adjuvant that may be administered to a subject (e.g., a patient), together with a compound of the present disclosure, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The amount administered depends on the compound formulation, route of administration, etc. and is generally empirically determined in routine trials, and variations will necessarily occur depending on the target, the host, and the route of administration, etc. Generally, the quantity of active compound in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The following are examples (Formulations 1-4) of capsule formulations.

Capsule Formulations

| Capsule | Formulation 1 | Formulation 2 | Formulation 3 | Formulation 4 |
|---|---|---|---|---|
| Formulation | mg/capsule | mg/capsule | mg/capsule | mg/capsule |
| Compound (solid | 100 | 400 | 400 | 200 |
| solution) | | | | |
| Silicon Dioxide | 0.625 | 2.5 | 3.75 | 1.875 |
| Magnesium | 0.125 | 0.5 | 0.125 | 0.625 |
| Stearate NF2 | | | | |
| Croscarmellose | 11.000 | 44.0 | 40.0 | 20.0 |
| Sodium NF | | | | |
| Pluronic F68 NF | 6.250 | 25.0 | 50.0 | 25.0 |
| Silicon Dioxide NF | 0.625 | 2.5 | 3.75 | 1.875 |
| Magnesium | 0.125 | 0.5 | 1.25 | 0.625 |
| Stearate NF | | | | |
| Total | 118.750 | 475.00 | 475.00 | 475.00 |
| Capsule Size | No. 4 | No. 0 | No. 0 | No. 2 |

### Preparation of Solid Solution

Crystalline compound (80 g/batch) and the povidone (NF K29/32 at 160 g/batch) are dissolved in methylene chloride (5000 mL). The solution is dried using a suitable solvent spray dryer and the residue reduced to fine particles by grinding. The powder is then passed through a 30 mesh screen and confirmed to be amorphous by x-ray analysis.

The solid solution, silicon dioxide and magnesium stearate are mixed in a suitable mixer for 10 minutes. The mixture is compacted using a suitable roller compactor and milled using a suitable mill fitted with 30 mesh screen. Croscarmellose sodium, Pluronic F68 and silicon dioxide are added to the milled mixture and mixed further for 10 minutes. A premix is made with magnesium stearate and equal portions of the mixture. The premix is added to the remainder of the mixture, mixed for 5 minutes and the mixture encapsulated in hard shell gelatin capsule shells.

### Use

In one aspect, methods for treating (e.g., controlling, relieving, ameliorating, alleviating, or slowing the progression of) or methods for preventing (e.g., delaying the onset of or reducing the risk of developing) one or more diseases, disorders, or conditions that have an anxiety, obsession and/or compulsive behavior component in a subject in need thereof are featured. The methods include administering to the subject an effective amount of a compound of formula (I) or (II) (and/or a compound of any of the other formulae described herein) or a salt (e.g., a pharmaceutically acceptable salt) thereof as defined anywhere herein to the subject.

In another aspect, the use of a compound of formula (I) or (II) (and/or a compound of any of the other formulae described herein) or a salt (e.g., a pharmaceutically acceptable salt) thereof as defined anywhere herein in the preparation of, or for use as, a medicament for the treatment (e.g., controlling, relieving, ameliorating, alleviating, or slowing the progression of) or prevention (e.g., delaying the onset of or reducing the risk of developing) of one or more diseases, disorders, or conditions that have an anxiety, obsession and/or compulsive behavior component is featured.

In embodiments, the one or more diseases, disorders, or conditions can include an anxiety disorder or neuropsychiatric disease, including but not limited to major depression, schizophrenia, autism, autism spectrum disorder, obsessive compulsive personality disorder, bipolar disorder, generalized anxiety disorder, social anxiety disorder, pediatric autoimmune neuropsychiatric disorders associated with streptococcal infections (PANDAS), pediatric acute-onset neuropsychiatric syndrome (PANS), anorexia nervosa, bulimia nervosa, Tourette syndrome, Asperger syndrome, body dysmorphic disorder, eating disorders, panic disorder, social phobia, Sydenham's chorea, Parkinson's disease, Huntington's disease, hoarding disorder, tic disorder, trichotillomania, dementia, Alzheimer's disease, attention deficit hyperactivity disorder, dermatillomania, onychophagia, and drug addiction.

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

In some embodiments, the compounds described herein can be coadministered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more compounds of formula (I) or (II) (including any subgenera or specific compounds thereof)). In other embodiments, these agents may be part of a single dosage form, mixed together with the compounds of the present disclosure in a single composition. In still another embodiment, these agents can be given as a separate dose that is administered at about the same time that one or more compounds of formula (I) or (II) (including any subgenera or specific compounds thereof) are administered (e.g., simultaneously with the administration of one or more compounds of formula (I) or (II) (including any subgenera or specific compounds thereof)). When the compositions of the present disclosure include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compositions of the present disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of the present disclosure may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in the present disclosure. Also within the present disclosure is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

The present disclosure will be further described in the following examples. It should be understood that these examples are for illustrative purposes only and are not to be construed as limiting the present disclosure in any manner. For example, one of ordinary skill will be able to exercise routine experimentation only, following the examples below (e.g., using the target-agnostic phenotypic screen and/or the LABORAS system in SAPAP3-deficient mice), to ascertain compounds that have efficacy in treating or preventing diseases, disorders, or conditions that have an anxiety, obsession and/or compulsive behavior component. One of ordinary skill will also be able to design and test additional compounds based on the P5C6 core by, e.g., making one or more substitutions thereto, based on principles of medicinal chemistry and pharmaceutical chemistry, again, using routine experimentation only.

### EXAMPLES

### Example 1. Discovery of P5C6 Using a Target-Agnostic In Vivo Phenotypic Screen

An In Vivo Discovery Approach:

A small molecule named “P5C6” has been identified as useful for designing new drugs and molecular probes to treat and study compulsive behavior. P5C6 was discovered through a non-conventional target-agnostic phenotypic screen in an animal model of OCD: SAPAP3-deficient mice. Traditionally, drug discovery proceeds through target-driven research programs that start with specific hypotheses regarding enzymes, receptors or channels already implicated in the disease. Biochemical assays that interrogate their function are then implemented in screening programs of small molecule modulators, and active small molecules that emerge from the screen are subsequently profiled in cell culture, animal models of disease, and ultimately in human patients. While targeted proteins and pathways reflect the current understanding of specific diseases, they also necessarily introduce an element of bias into discovery efforts. In this context, in vivo phenotypic screening strategies offer an attractive alternative approach to drug discovery.

Specifically, the phenotypic screening involves evaluating small molecules for efficacy at an organismal level⁵. These assays return compounds with a desired biological outcome, such as behavioral phenotype, without bias towards presumed mechanisms of action. Phenotypic screens can prove advantageous when no consensus exists regarding suitable biological targets, or when investigators value early efficacy and safety over immediate scientific understanding of mechanism of action. While uncertainty surrounding the mechanism of action presents a challenge, several compensating considerations can favor phenotypic screening strategies. First, hits that are identified cause a desired biological outcome rather than simply binding or inhibiting a specific target in vitro. This presents a relative advantage compared to target-driven approaches involving unvalidated targets. Second, for compounds to score as hits, they must have suitable physical properties to engage their targets within the cellular or organismal milieu, display suitable toxicity profiles, and remain chemically stable in the context of the experiment. These features facilitate transition into rigorous testing in more sophisticated preclinical animal models. Finally, determining the mechanism of action of newbiologically active small molecules may reveal previously unanticipated protein targets and biochemical pathways relevant to understanding and treating disease.

Target-Agnostic In Vivo Phenotypic Screening in SAPAP3-Deficient Mice has Identified a Molecule that Normalizes Grooming:

Investigations into synapse-associated protein 90/postsynaptic density protein 95-associated protein 3 (SAPAP3) have provided important insights into the pathophysiology of OCD. Genetic variations in SAPAP3 have been identified in some patients with OC-spectrum disorders, such as trichotillomania^{1,2,3}, and SAPAP3 is enriched in the striatum where it influences excitatory synaptic function⁴. Importantly, SAPAP3-deficient mice display compulsive grooming leading to acquisition of facial lesions, which is ameliorated by fluoxetine, the most common treatment for patients with OCD⁴. Thus, SAPAP3-deficient mice are a valuable model of compulsive behavior in OCD. We used these mice to implement an in vivo phenotypic screen to identify new small, drug-like molecules capable of ameliorating compulsive grooming, and identified efficacious small molecules described herein, which are referred to as “P5C6” class of molecules. Synthetic organic chemistry can be used to modify these molecules to increase potency and efficacy, improve penetration of the blood-brain-barrier, and also to generate molecular probes to elucidate their mechanism of action. Ultimate elucidation of their mechanism of action can provide new insight into the molecular mechanisms underlying pathologically compulsive behavior.

By screening from a set of carefully selected 1000 compounds, we identified a unique molecule that normalized grooming in SAPAP3-deficient mice. All molecules were initially pooled into groups of 10, and were infused directly into the left lateral ventricle of four adult (14 weeks of age) male SAPAP3-deficient mice for 7 days by means of surgically implanted Alzet osmotic minipumps. Grooming behavior was assessed on the final day of compound infusion using a spray-induced grooming assay that we have standardized and developed.

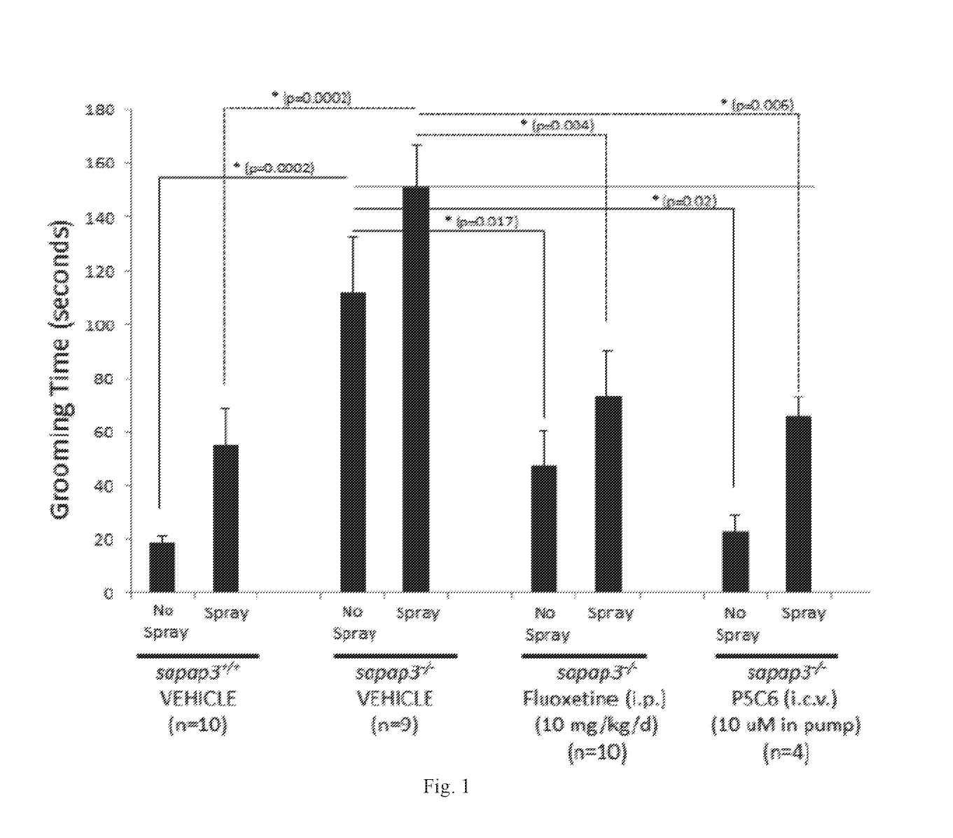

Briefly, between 0800 and 1700 hours, test animals were acclimated for 30 min in clear housing and then exposed to a small water spray bottle without being sprayed, followed by video recording for 5 min. Immediately following this period, test animals were sprayed 4 times with water, with the same spray bottle, near the head to induce grooming behavior, and again videotaped for 5 min. This method provided a means for rapid quantification to compare baseline grooming to induced grooming. Time spent grooming was then manually determined with a stopwatch while observing the video, with the observer blind to genotype and treatment group. The number of grooming bouts was also manually determined by observing the video. As shown in FIG. 1, both baseline and spray-induced grooming are significantly greater in SAPAP3-deficient mice than wild type littermates, and both modalities of grooming are restored to wild type levels in SAPAP3-deficient mice by intraperitoneal delivery of fluoxetine (10 mg/kg/d). A similar protective effect was seen in mice administered pool #5, and subsequent evaluation of the efficacy of the ten individual components of pool 5 showed that the 6^{th} (“P5C6”, structure shown below) compound normalized basal and spray-induced grooming in SAPAP3-deficient mice to wild type levels.

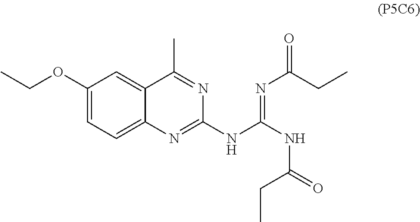 [SMILES: CCOc1ccc2nc(N/C(=N/C(=O)CC)NC(=O)CC)nc(C)c2c1]

P5C6 can be chemically optimized to generate new chemical matter that confers therapeutic benefits, as well as new molecular probes to elucidate the mechanisms of action of P5C6. This latter effort will provide new insight into the underlying pathophysiology of compulsive behavior in neuropsychiatric disease, like OCD and OC-spectrum disorders.

### Example 2: Exemplary P5C6 Analogs

Synthetic chemistry can be used to prepare a modified quinazoline P5C6 core structure bearing organic functional groups that can be readily diversified to a variety of analogs. From this core structure, new compounds can be synthesized and assayed for increased potency and efficacy, as well as improved ability to penetrate the brain. Compounds can be generated and systematically evaluated in a rodent model of pathologically compulsive behavior such as the SAPAP3-deficient mice. Molecular probes can also be generated to elucidate the P5C6 mechanism of action.

Specific Chemical Modification of P5C6:

In order to understand the mode of action of P5C6, and to optimize their bioactivities, a variety of modified drugs are needed for biological evaluation in the phenotypic screen. Synthetic organic chemistry can be used to produce these new compounds. P5C6 is one of a class of quinazolines that have been previously examined as adenosine receptor antagonists⁷. The heterocyclic quinazoline core of P5C6 derives from substituted aniline, of which there are many commercially available variants. This facilitates chemical modifications to the molecular structure, particularly at positions 5-8 of the quinazoline.

Preparing Versatile Core Structures for Combinatorial Synthesis:

The precedented methodology in synthetic chemistry can be rapidly exploited in order to synthesize the initial sets of new compounds. New methodology may eventually be developed. To facilitate synthesis, versatile core structures can be prepared to allow for convenient derivatization via combinatorial approaches.

Structural Diversity from the Core of P5C6:

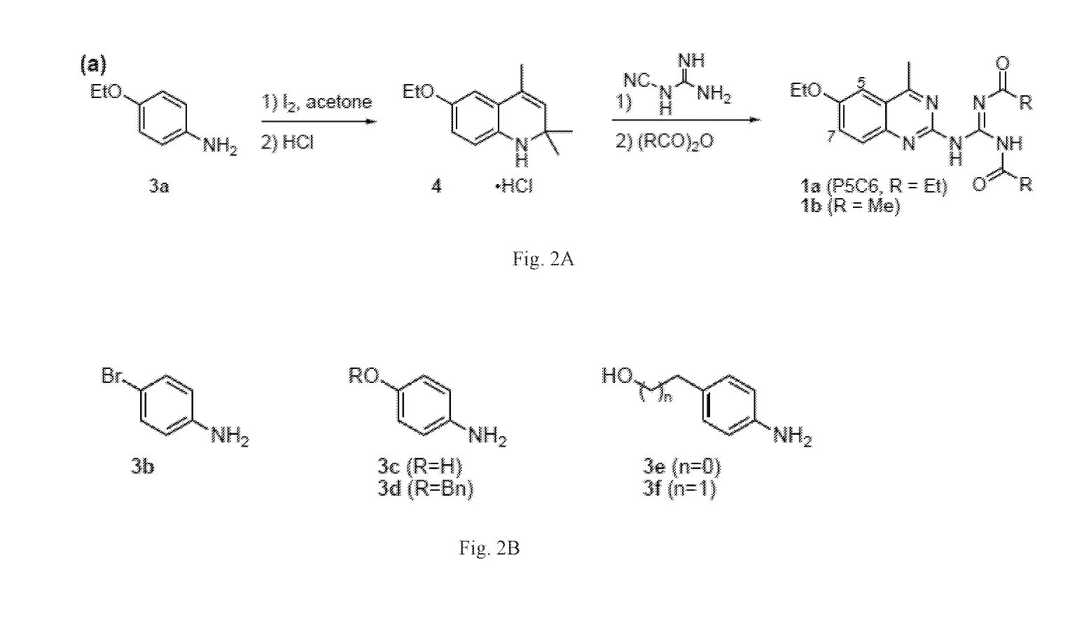

To access the core structure of P5C6, a known synthetic route (FIG. 2A) is first exploited and analogs that bear synthetic handles, such as Br, OH, or (CH2)nOH groups are prepared. The validated synthetic method starts with p-ethoxyaniline (3a), and incorporates two equivalents of acetone within dihydroquinoline 4, also known as “acetoneanil”. Upon treatment of 4 with dicyandiamide (cyanoguanidine), the nitrile group is incorporated into the ring to produce the quinazoline ring system, and acylation yields 1 (e.g., 1a, 1b). Various anilines may be used (FIG. 2B), providing the first point of structural diversification. The methyl substituent of the quinazoline derives from acetone, which provides a second point of diversification with different ketones, leading to a variety of 3- and 4-substituted analogs. Finally, numerous acylating agents can be applied in the last step to afford diacylguanidines (various R groups, FIG. 2A), as a third point of diversification. In some embodiments, the R group in FIG. 2A can be, at each occurrence, independently selected from C₁₋₁₂ alkyl, C₂₋₁₂ alkenyl or C₂₋₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, oxo, C₁₋₆ alkyl carbonyl, C₁₋₆ alkoxycarbonyl, cyano, nitro and/or amine.

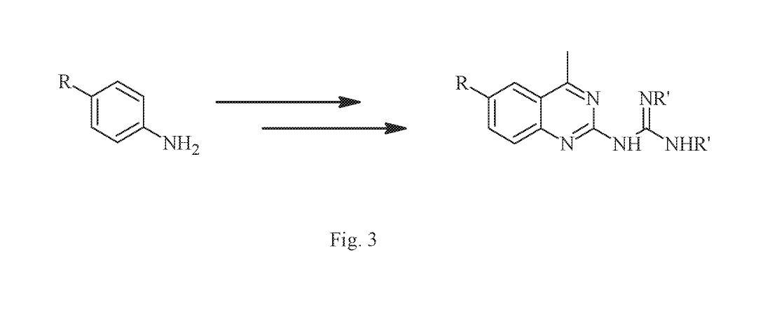

In certain embodiments, a new series of quinazolin-2-yl-guanidines from readily available commercial anilines are prepared as shown in FIG. 3. Incorporating functionalities into the starting aniline leads to a series of new quinazolin-2-yl-guanidines with variations at the 6-position, including halide, alkyl and alkoxy groups, as well as alternative substitution patterns. In some examples, the R group in FIG. 3 can be selected from hydrogen; halo; hydroxyl; C₁₋₆ (e.g., C₁₋₃) alkoxyl optionally substituted with 1 or more hydroxyl, cyano and/or halo; C₁₋₆ (e.g., C₁₋₃) alkyl carbonyl; C₁₋₆ (e.g., C₁₋₃) alkoxycarbonyl; cyano; nitro; amine; R^{A} and R^{B}. R^{A} is selected from C₁₋₆ (e.g., C₁₋₃) alkyl, C₂₋₆ (e.g., C₂₋₃) alkenyl and C₂₋₆ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) alkyl carbonyl, C₁₋₆ (e.g., C₁₋₃) alkoxycarbonyl, cyano, nitro, and/or amine. R^{B} is selected from C₃₋₁₂ cycloalkyl, C₂₋₆ heterocyclyl, C₆₋₁₂ aryl and C₄₋₁₂ heteroaryl, each optionally substituted with 1 or more halo; hydroxyl; C₁₋₆ alkyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ alkoxyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₄₋₁₂ heteroaryl, and/or amine; C₂₋₆ (e.g., C₂₋₃) alkoxycarbonyl; cyano; nitro; azide; amine; C₃₋₁₂ cycloalkyl; C₂₋₆ heterocyclyl, C₆₋₁₂ aryl; and/or C₄₋₁₂ heteroaryl; wherein each of the substituents C₃₋₁₂ cycloalkyl, C₂₋₆ heterocyclyl, C₆₋₁₂ aryl and C₄₋₁₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) alkoxycarbonyl, cyano, nitro and/or amine. The R′ group in FIG. 3 can be hydrogen or C(O)R″ where R″ can be, at each occurrence, independently selected from C₁-C₁₂ alkyl, C₂-C₁₂ alkenyl or C₂-C₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, oxo, C₁₋₆ alkyl carbonyl, C₁₋₆ alkoxycarbonyl, cyano, nitro and/or amine.

Improvement of the Initial Hit (P5C6) from the Screen:

With P5C6 core structure in hand, it is possible to next improve their potency and efficacy by preparing and evaluating analogs by standard medicinal chemistry approaches. Issues of drug stability in vivo and transfer across the blood-brain barrier (BBB) are priorities for analog design and synthesis. Specific structure features associated with in vivo stability and BBB transit (described below) are incorporated into analogs of P5C6 using organic synthesis.

Oxidative Degradation In Vivo.

To promote useful drug half-lives in vivo, a problem may need to be addressed: the presence of relatively electron-rich aromatic ring (p-ethoxyaniline substructure of P5C6) may render them excessively prone to oxidative processes in vivo. In the synthetic schemes presented herein, these substructures can be exchanged for spatially similar building blocks bearing electron-withdrawing substituents such as F, CF3, acyl, or sulfonyl (e.g., alkylsulfonyl). The convergent synthesis from simple building blocks can readily allow for incorporation of such substituents in the precursors.

Transfer Across the Blood-Brain-Barrier.

Access to the brain in vivo requires attention to structural features that may permit the compounds to cross the blood-brain barrier (BBB). There are a wide range of possibilities here, including incorporation of the drug candidate within engineered nanoparticles⁸, linkage of the drug to glucose, phenylalanine or tyrosine⁹, or adjusting the lipophilicity. Initially, analogs can be prepared that adjust the lipophilicity close to a log P=2, which is known to enhance the crossing of the BBB¹⁰. For P5C6, the modular synthesis design allows that the three ethyl groups may be exchanged for methyl groups to lower the computationally estimated log P from 2.68 to 1.42 (and points between). Concentration of P5C6 analogs in blood and brain tissue can be analyzed after IP and PO administration via tandem liquid chromatography mass spectrometry assays. Half-life of the molecules can also be assayed using conventional methods known in the art.

Preparing Bioconjugates for Mechanism of Action Studies:

With improved P5C6 analogs available, bioconjugates for mode of action studies can then be prepared. Using the phenotypic screen for biological evaluation, analogs containing a simple alkyne-containing linker in various locations can be used to identify a suitable location for the linker that preserves biologic activity. Then, azide-alkyne click chemistry can be employed for linkage to commercially available azide derivatives of biotin, photocrosslinkers, dyes, and other tools for mechanism of action studies.

Bioconjugates of P5C6:

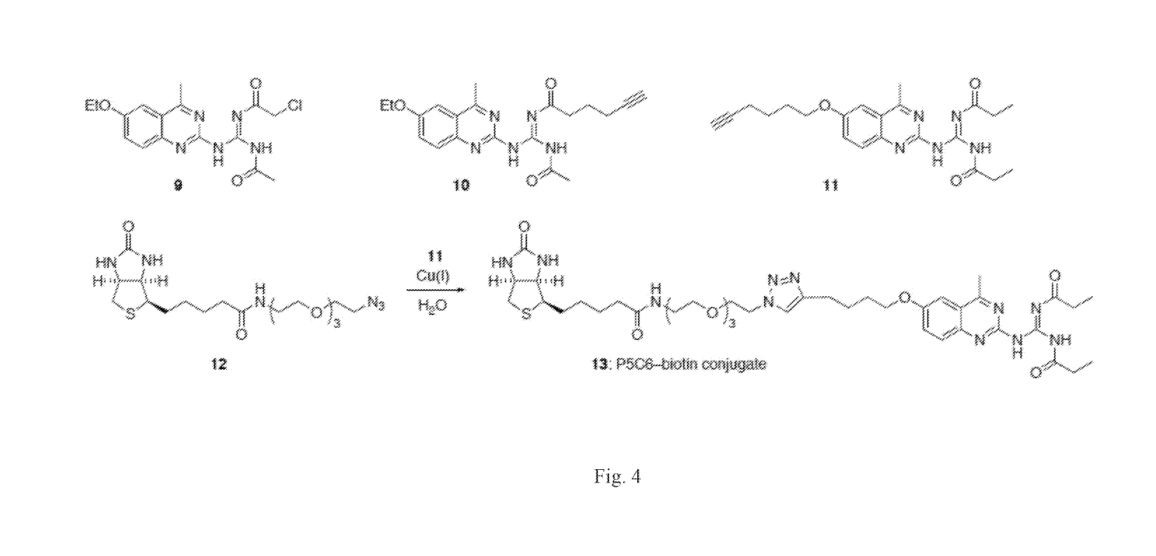

The guanidine acylation step can be modified by executing acylation with limiting amounts of chloroacetic anhydride or 5-hexynoyl chloride, followed by completing the second acylation with acetic anhydride. The chloroacetamide 9 (FIG. 4) produced in this fashion can present a synthetic handle for nucleophilic substitution with thiol derivatives of biotin or other biomolecules or fluorescent tags. The hexynoyl analog 10 can be prepared in similar fashion, and engaged in Cu-catalyzed aqueous azide-alkyne click chemistry with azide derivatives of biotin or other biomolecules or fluorescent tags. Alkynyl units can also be incorporated in the 6-alkoxy substituent (e.g., 11) via substitution of the corresponding 6-bromoquinazoline with 5-hexyn-1-ol, or by substitution at an earlier stage of synthesis. Once it is confirmed that bioactivity is retained, any of these alkynes may then be transformed to various bioconjugates using click chemistry with azides; for example, ligation with commercially available azide-substituted biotin derivative 12 would furnish triazole 13, a P5C6-biotin conjugate. In the event that the aforementioned derivatives fail to maintain sufficient activity, selective oxidation of the benzylic position of the 4-methyl substituent may be attempted using radical halogenation or transition metal-catalyzed C—H activation. Finally, it should be noted that each of these strategies accommodates variations to intervening linkers such as alkyl chains, polyethylene glycol chains, and the like, of various spacer lengths as needed to maintain bioactivity.

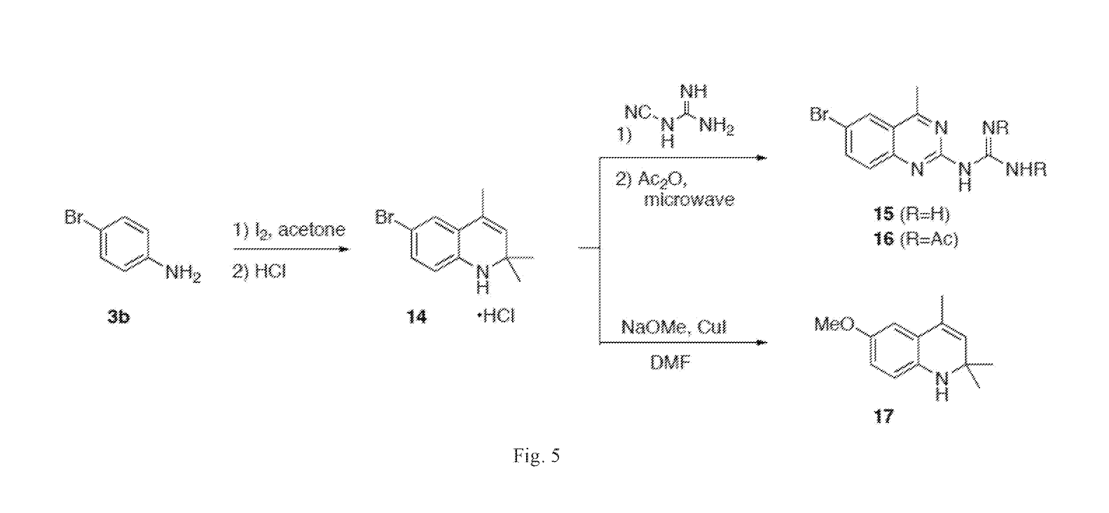

Studies on P5C6 have confirmed the viability of the synthetic route using a Br substituent in place of the OEt group. Thus, using the sequence outlined in FIG. 2A, the 6-bromo dihydroquinoline 14 was prepared in multigram quantity, and from this, analogs 15 and 16 have been prepared (FIG. 5). Secondly, the Br substituent may be replaced with an alkoxy group via Cucatalyzed coupling at the acetoneanil stage, affording compound 17.

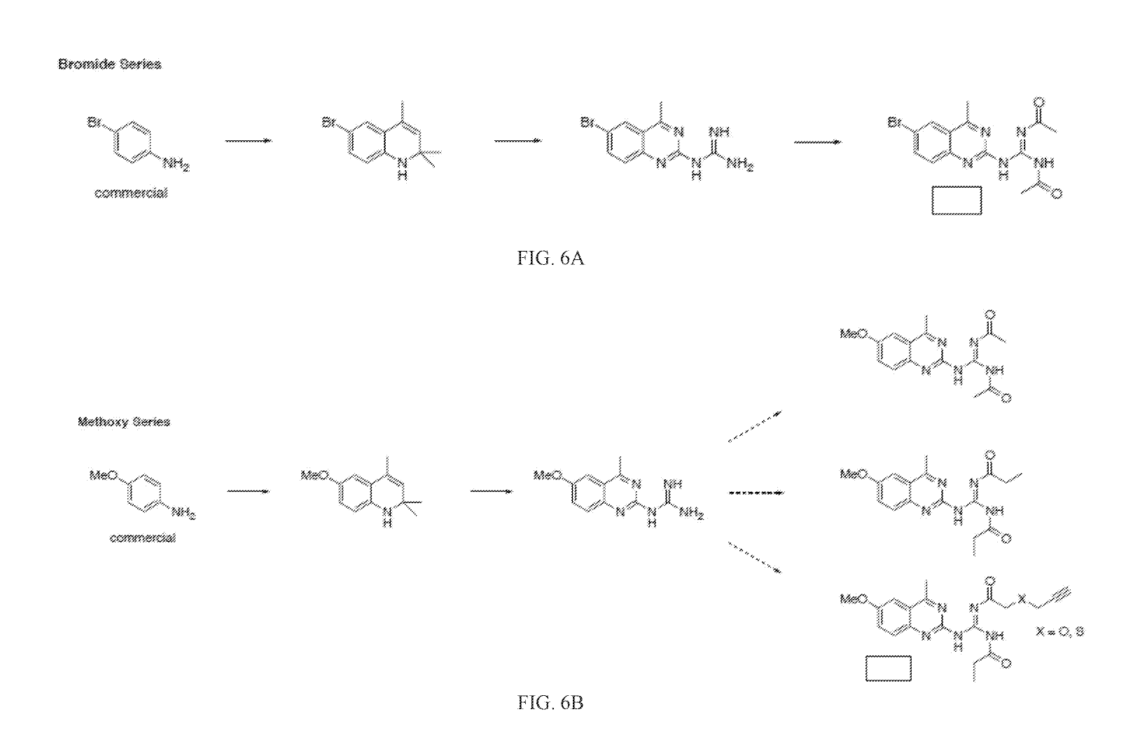

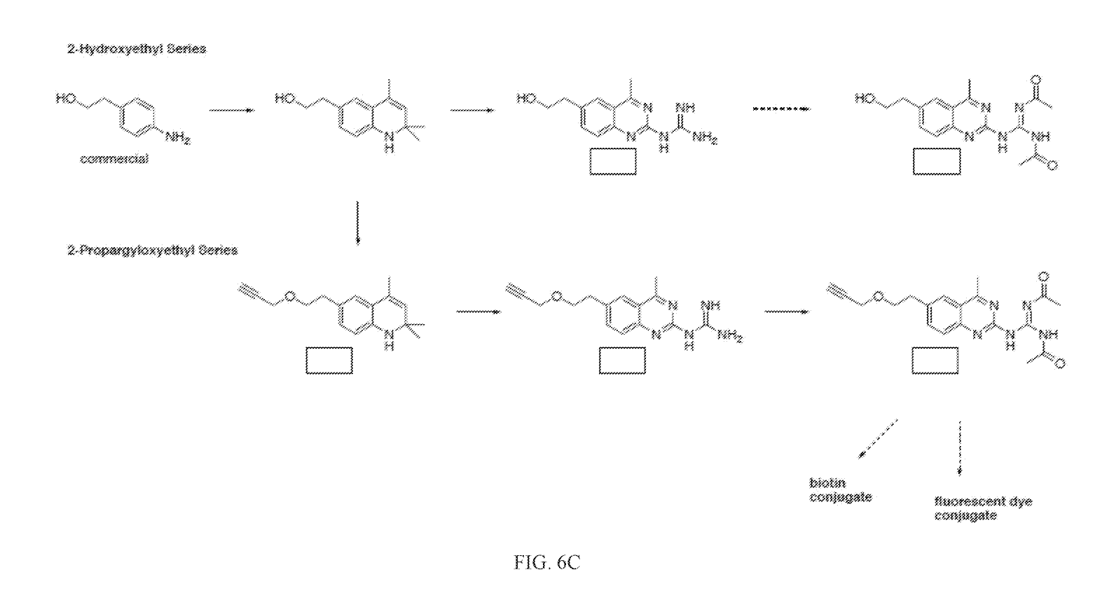

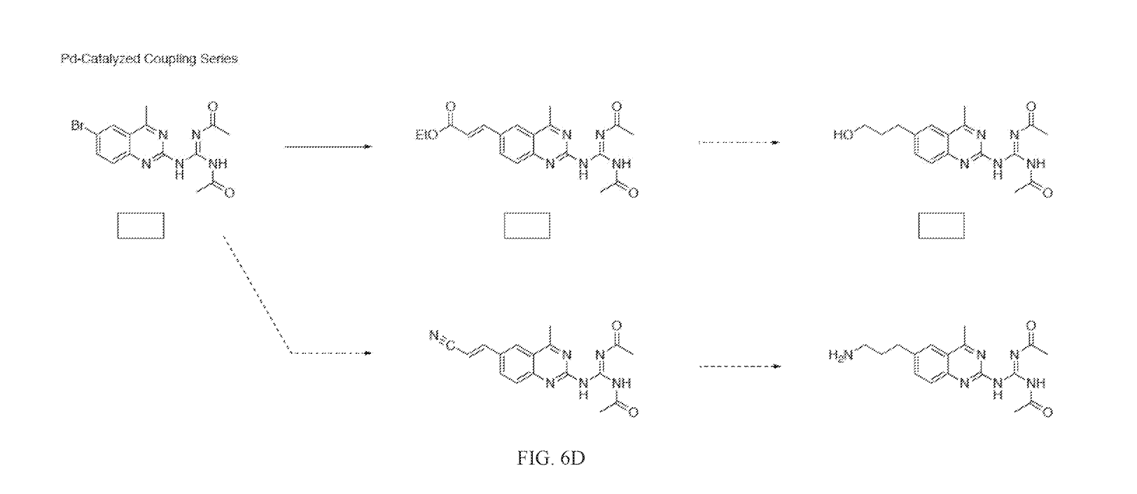

Additional exemplary P5C6 analogs and representative synthetic routes are shown in FIGS. 6A-6D.

Biologic Evaluation of Efficacy of Novel Chemical Variants of P5C6:

Since conducting the initial screen that identified the biologic activity of P5C6, more sophisticated equipment has been obtained to allow automated monitoring of grooming in mice. Moving forward, all grooming behavior can thus be assayed in 4 male and 4 female 14 week old SAPAP3-deficient mice by means of the LABORAS system that allows 24 hour automated collection of nonstimulated, basal grooming using a vibration sensitive plate to monitor fine motor activity. It has been established that this system is consistent with the spray test in terms of quantifying relative genotype and treatment-specific differences in grooming in SAPAP3-deficient mice. Thus, all test compounds generated can be evaluated for efficacy in restoring normal grooming to SAPAP3-deficient mice in this more rigorous and automated testing paradigm.

Briefly, LABORAS (Metris) is a system that uses a carbon fiber plate to detect behavior-specific vibration patterns created by animals. Various behavioral parameters are determined by LABORAS software processing of the vibration pattern. Data are collected uninterrupted over a 24-h period, enabling comprehensive quantification of basal grooming time, bouts and locomotor activity in the home cage environment throughout the lightdark cycle. Before data collection, test animals are acclimated in the test room for 1 wk. Then, test animals are placed in a standard cage atop the carbon fiber platforms. Vibrations are recorded for 24 hours, and then the animals are removed. Vibration data are processed via LABORAS 2 software. Further assessment of locomotor activity as distance in meters traveled is also automated, and has shown that changes in grooming in SAPAP3-deficient mice are not due to an effect on locomotor activity. This parameter can also be monitored in the evaluation of efficacy of new chemicals generated.

The P5C6 series of compounds can help develop new treatments for patients suffering from pathologically compulsive behavior, and also to generate novel molecular tools to help elucidate the underlying mechanisms of these disorders.

### REFERENCES

1. Boardman, L., van der Merwe, L., Lochner, C., Kinnear, C. J., Seedat, S., Stein, D. J., Moolman-Smook, J. C. and Hemmings, S. M. (2011). Investigating SAPAP3 variants in the etiology of obsessive-compulsive disorder and trichotillomania in the South African white population. Compr Psychiatry 52: 181-7.
2. Zuchner, S., Wendland, J. R., Ashley-Kock, A. E., Collins, A. L., Tran-Viet, K. N., Quinn, K., Timpano, K. C., Cuccaro, M. L., Perical-Vance, M. A., Steffens, D. C., Krishnan, K. R., Feng, G. and Murphy, D. L. (2009). Multiple rare SAPAP3 missense variants in trichotillomania and OCD. Mol Psychiatry 14: 6-9.
3. Bienvenu, O. J., Wang, Y., Shugart, Y. Y., Welch, J. M., Grados, M. A., Fyer, A. J., Rauch, S. L., McCracken, J. T., Rasmussen, S. A., Murphy, D. L., Cullen, B., Valle, D., Hoehn-Saric, R., Greenberg, B. D., Pinto, A., Knowles, J. A., Piacentini, J., Pauls, D. L., Liang, K. Y., Wilbur, V. L., Riddle, M., Samuels, J. F., Feng, G. and Nestady, G. (2009). Sapap3 and pathological grooming in humans: Results from the OCD collaborative genetics study. Am J Med Genet Neuropsychiatr Genet 150B: 710-20.
4. Welch, J. M., Lu, J., Rodriguiz, R. M., Trona, N. C., Peca, J., Ding, J. D., Feliciano, C., Chen, M., Adams, J. P., Luo, J., Dudek, S. M., Weinberg, R. J., Calakos, N., Wetsel, W. C. and Feng, G. (2007). Cortico-striatal synaptic defects and OCD-like behaviours in Sapap3-mutant mice. Nature 448: 894-900.
5. D. C. Swinney, J. Anthony, Nature Rev. Drug Discov. 2011, 10, 507. (b) J. A. Lee, M. T. Uhlik, C. M. Moxham, D. Tomandl, D. J. Sall, J. Med. Chem. 2012, 55, 4527.
6. Xu, P., Grueter, B. A., Britt, J. K., McDaniel, L., Huntington, P. J., Hodge, R., Tran, S., Mason, B. L., Lee, C., Vong, L., Lowell, B. B., Malenka, R.*, Lutter, M.* and Pieper, A. A.* (2013). Double deletion of melanocortin 4 receptors and SAPAP3 corrects compulsive behavior and obesity in mice. Proc Natl Acad Sci USA 110: 10759-64.
7. Webb, T. R.; Lvovskiy, D.; Kim, S.-A.; Ji, X.; Melman, N.; Linden, J.; Jacobson, K. A. (2003). Quinazolines as Adenosine Receptor Antagonists: SAR and Selectivity for A2B Receptors. Bioorg. Med. Chem. 11: 77-85.
8. (a) Rocha, S. (2013). Targeted Drug Delivery Across the Blood Brain Barrier in Alzheimer's Disease. Curr. Pharm. Design 19: 6635-6646. (b) Lai, F.; Fadda, A. M.; Sinico, C. (2013). Liposomes for brain delivery. Exp. Opin. Drug Deliv. 10: 1003-1022. (c) Daniels, T. R.; Bernabeu, E.; Rodriguez, J. A.; Patel, S.; Kozman, M.; Chiappetta, D. A.; Holler, E.; Ljubimova, J. Y.; Helguera, G.; Penichet, M. L. (2012) Biochim. Biophys. Acta, Gen. Subj. 1820: 291.
9. Punitha, A. D.; Srivastava, A. K. (2013). CNS drug targeting: have we travelled in right path? J. Drug Target 21: 787-800.
10. Lipinski, C. A.; Lombardo, F.; Dominy, B. W.; Feeney, P. J. (1997). Experimental and computational approaches to estimate solubility and permeability in drug discovery and development settings. Adv. Drug Deliv. Rev. 23: 3-25

### EQUIVALENTS

The present disclosure provides among other things novel methods and compositions for treatment of anxiety and/or compulsive behavior. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and patent applications referenced in this specification are incorporated herein by reference in their entirety for all purposes to the same extent as if each individual publication, patent or patent application were specifically indicated to be so incorporated by reference.

1. A compound of formula (I) or (II), or a pharmaceutically acceptable salt or prodrug thereof: 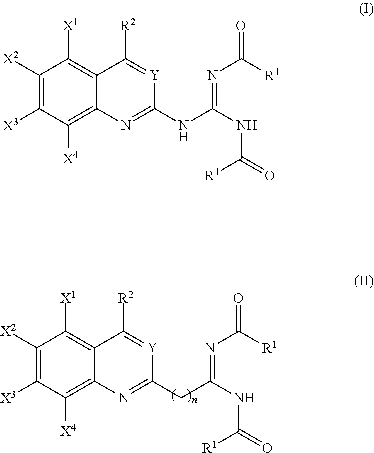
wherein:
X¹, X², X³, and X⁴ are each independently selected from: hydrogen; halo; hydroxyl; C₁₋₆ (e.g., C₁₋₃) alkoxyl optionally substituted with 1 or more hydroxyl, cyano and/or halo; C₁₋₆ (e.g., C₁₋₃) alkyl carbonyl; C₁₋₆ (e.g., C₁₋₃) alkoxycarbonyl; cyano; nitro; amine; R^{A} and R^{B};
wherein R^{A} at each occurrence is independently selected from C₁₋₆ (e.g., C₁₋₃) alkyl, C₂₋₆ (e.g., C₂₋₃) alkenyl and C₂₋₆ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) thioalkoxyl, C₁₋₆ (e.g., C₁₋₃) alkyl carbonyl, C₁₋₆ (e.g., C₁₋₃) alkoxycarbonyl, cyano, nitro, and/or amine;
wherein R^{B} at each occurrence is independently selected from C₃₋₁₂ cycloalkyl, C₂₋₆ heterocyclyl, C₆₋₁₂ aryl and C₄₋₁₂ heteroaryl, each optionally substituted with 1 or more halo; hydroxyl; C₁₋₆ alkyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₄₋₁₂ heteroaryl, and/or amine; C₂₋₆ (e.g., C₂₋₃) alkoxycarbonyl; cyano; nitro; azide; amine; C₃₋₁₂ cycloalkyl; C₂₋₆ heterocyclyl; C₆₋₁₂ aryl; and/or C₄₋₁₂ heteroaryl; wherein each of the substituents C₃₋₁₂ cycloalkyl, C₂₋₆ heterocyclyl, C₆₋₁₂ aryl and C₄₋₁₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) thioalkoxyl, C₁₋₆ (e.g., C₁₋₃) alkoxycarbonyl, cyano, nitro and/or amine;
R¹ and R² at each occurrence, are each independently selected from C₁₋₁₂ alkyl, C₂₋₁₂ alkenyl or C₂₋₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆ alkoxycarbonyl, cyano, nitro and/or amine; wherein the C₁₋₆ alkoxyl, C₁₋₆ thioalkoxyl, C₁₋₆ alkyl carbonyl, and C₁₋₆ alkoxycarbonyl groups each are optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆ alkoxycarbonyl, cyano, nitro and/or amine;
Y is CH or N; and
n is an integral selected from 1-12.

2. The compound or salt or prodrug of claim 1, wherein at least one of X¹, X², X³, and X⁴ is not hydrogen.

3. The compound or salt or prodrug of claim 1, wherein X² is not hydrogen.

4. The compound or salt or prodrug of claim 1, selected from:

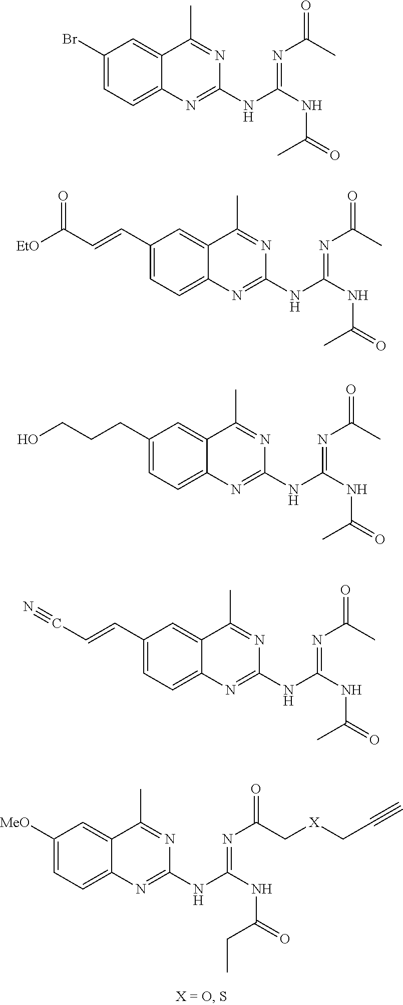

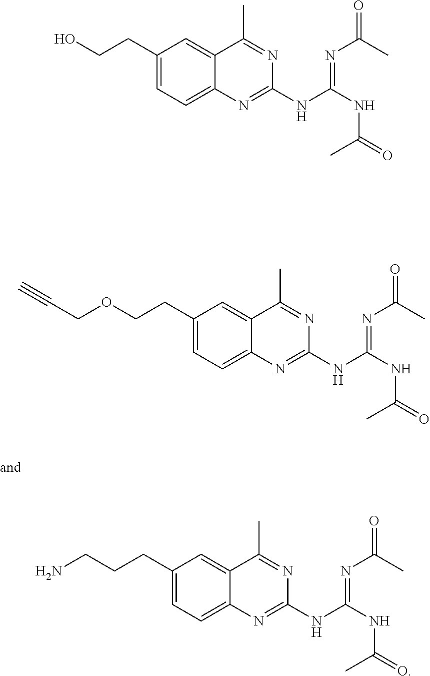 [SMILES: CC(=O)/N=C(\NC(C)=O)Nc1nc(C)c2cc(CCO)ccc2n1 ; C#CCOCCc1ccc2nc(N/C(=N/C(C)=O)NC(C)=O)nc(C)c2c1 ; CC(=O)/N=C(\NC(C)=O)Nc1nc(C)c2cc(CCCN)ccc2n1]

5. The compound or salt or prodrug of any one of claims 1-3, wherein the compound is not

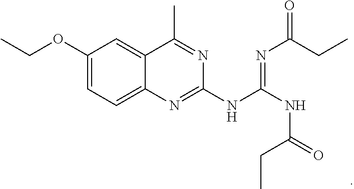 [SMILES: CCOc1ccc2nc(N/C(=N/C(=O)CC)NC(=O)CC)nc(C)c2c1]

6. The compound or salt or prodrug of any one of claims 1-4, for use in the treatment of anxiety disorder or a neuropsychiatric disease having an obsession and/or pathologically compulsive behavior component.

7. The compound or salt or prodrug of claim 5, wherein the anxiety disorder or neuropsychiatric disease is selected from major depression, schizophrenia, autism, autism spectrum disorder, obsessive compulsive personality disorder, bipolar disorder, generalized anxiety disorder, social anxiety disorder, pediatric autoimmune neuropsychiatric disorders associated with streptococcal infections (PANDAS), pediatric acute-onset neuropsychiatric syndrome (PANS), anorexia nervosa, bulimia nervosa, Tourette syndrome, Asperger syndrome, body dysmorphic disorder, eating disorders, panic disorder, social phobia, Sydenham's chorea, Parkinson's disease, Huntington's disease, hoarding disorder, tic disorder, trichotillomania, dementia, Alzheimer's disease, attention deficit hyperactivity disorder, dermatillomania, onychophagia, and drug addiction.

8. The compound or salt or prodrug of any one of claims 1-4 for use in the manufacture of a medicament for the treatment of anxiety disorder or a neuropsychiatric disease having an obsession and/or pathologically compulsive behavior component.

9. The compound or salt or prodrug of any one of claims 6-8, wherein the compound is

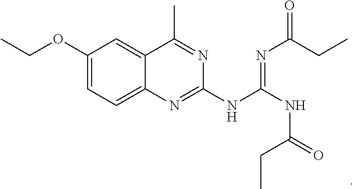 [SMILES: CCOc1ccc2nc(N/C(=N/C(=O)CC)NC(=O)CC)nc(C)c2c1]

10. A pharmaceutical composition comprising the compound or salt or prodrug of any one of claims 1-5, and a pharmaceutically acceptable carrier.

11. A method for treating anxiety disorder or a neuropsychiatric disease having an obsession and/or pathologically compulsive behavior component, comprising administering an effective amount of a compound of formula (I) or (II) or a pharmaceutically acceptable salt or prodrug thereof, to a patient in need thereof: 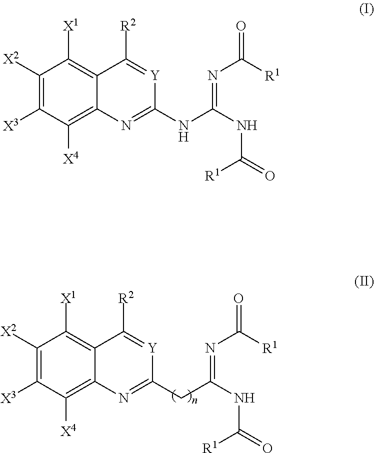
wherein:
X¹, X², X³, and X⁴ are each independently selected from: hydrogen; halo; hydroxyl; C₁₋₆ (e.g., C₁₋₃) alkoxyl optionally substituted with 1 or more hydroxyl, cyano and/or halo; C₁₋₆ (e.g., C₁₋₃) alkyl carbonyl; C₁₋₆ (e.g., C₁₋₃) alkoxycarbonyl; cyano; nitro; amine; R^{A} and R^{B};
wherein R^{A} at each occurrence is independently selected from C₁₋₆ (e.g., C₁₋₃) alkyl, C₂₋₆ (e.g., C₂₋₃) alkenyl and C₂₋₆ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) thioalkoxyl, C₁₋₆ (e.g., C₁₋₃) alkyl carbonyl, C₁₋₆ (e.g., C₁₋₃) alkoxycarbonyl, cyano, nitro, and/or amine;
wherein R^{B} at each occurrence is independently selected from C₃₋₁₂ cycloalkyl, C₂₋₆ heterocyclyl, C₆₋₁₂ aryl and C₄₋₁₂ heteroaryl, each optionally substituted with 1 or more halo; hydroxyl; C₁₋₆ alkyl optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₄₋₁₂ heteroaryl, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₄₋₁₂ heteroaryl, and/or amine; C₂₋₆ (e.g., C₂₋₃) alkoxycarbonyl; cyano; nitro; azide; amine; C₃₋₁₂ cycloalkyl; C₂₋₆ heterocyclyl; C₆₋₁₂ aryl; and/or C₄₋₁₂ heteroaryl; wherein each of the substituents C₃₋₁₂ cycloalkyl, C₂₋₆ heterocyclyl, C₆₋₁₂ aryl and C₄₋₁₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) thioalkoxyl, C₁₋₆ (e.g., C₁₋₃) alkoxycarbonyl, cyano, nitro and/or amine;
R¹ and R² at each occurrence, are each independently selected from C₁₋₁₂ alkyl, C₂₋₁₂ alkenyl or C₂₋₁₂ alkynyl, each optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆alkoxycarbonyl, cyano, nitro and/or amine; wherein the C₁₋₆ alkoxyl, C₁₋₆ thioalkoxyl, C₁₋₆ alkyl carbonyl, and C₁₋₆ alkoxycarbonyl groups each are optionally substituted with 1 or more C₁₋₆ alkyl, C₂₋₆ alkenyl, C₂₋₆ alkynyl, halo, hydroxyl, C₁₋₆ alkoxyl, C₁₋₆ thioalkoxyl, oxo, C₁₋₆ alkyl carbonyl, C₁₋₆alkoxycarbonyl, cyano, nitro and/or amine;
Y is CH or N; and
n is an integral selected from 1-12.

12. The method of claim 11, wherein at least one of X¹, X², X³, and X⁴ is not hydrogen.

13. The method of claim 11, wherein X² is not hydrogen.

14. The method of claim 11, wherein the compound is selected from:

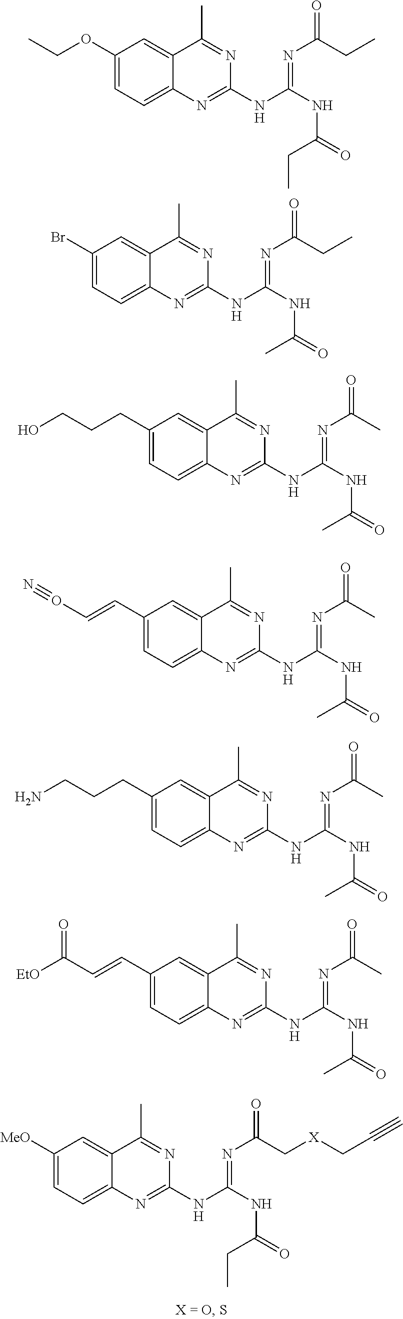

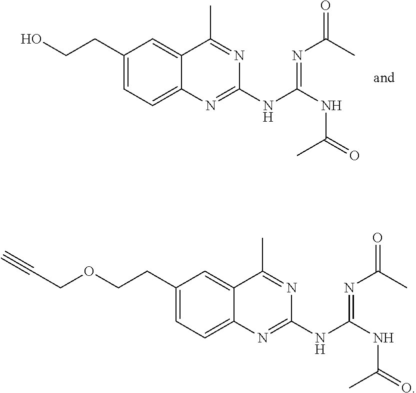 [SMILES: CC(=O)/N=C(\NC(C)=O)Nc1nc(C)c2cc(CCO)ccc2n1 ; C#CCOCCc1ccc2nc(N/C(=N/C(C)=O)NC(C)=O)nc(C)c2c1]

15. The method of any one of claims 11-13, wherein the compound is

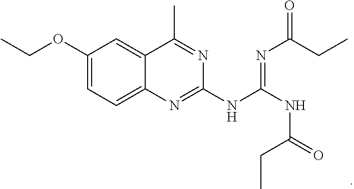 [SMILES: CCOc1ccc2nc(N/C(=N/C(=O)CC)NC(=O)CC)nc(C)c2c1]

16. The method of any one of claims 11-15, wherein the anxiety disorder or neuropsychiatric disease is selected from major depression, schizophrenia, autism, autism spectrum disorder, obsessive compulsive personality disorder, bipolar disorder, generalized anxiety disorder, social anxiety disorder, pediatric autoimmune neuropsychiatric disorders associated with streptococcal infections (PANDAS), pediatric acute-onset neuropsychiatric syndrome (PANS), anorexia nervosa, bulimia nervosa, Tourette syndrome, Asperger syndrome, body dysmorphic disorder, eating disorders, panic disorder, social phobia, Sydenham's chorea, Parkinson's disease, Huntington's disease, hoarding disorder, tic disorder, trichotillomania, dementia, Alzheimer's disease, attention deficit hyperactivity disorder, dermatillomania, onychophagia, and drug addiction.
